(19) United States
(12) Patent Application Publication
(10) Pub. No.: US 2009/0235936 A1
(43) Pub. Date: Sep. 24, 2009

(54) Valved Fenestrated Tracheotomy Tube Having Inner and Outer Cannulae with Pressure Relief

(75) Inventor: Eric D. Blom, Carmel, IN (US)

Correspondence Address: BARNES & THORNBURG LLP, 11 SOUTH MERIDIAN, INDIANAPOLIS, IN, 46204, US

(73) Assignee: HANSA MEDICAL PRODUCTS, INC., Carmel, IN (US)

(21) Appl. No.: 12/050,525

(22) Filed: Mar. 18, 2008

(51) Int. Cl. A61M 16/20; A62B 9/02

(52) U.S. Cl. 128/207.16

(57) ABSTRACT

An outer cannula has a first port for orienting outside the neck of a wearer, a second port for orienting within the trachea of the wearer, a first passageway coupling the first port to the second port to permit the flow of gases from the first port to the second during inhalation by the wearer and from the second port during exhalation by the wearer, and a third port between the first and second ports. An inner cannula is provided for insertion into the first passageway via the first port when the wearer desires to be able to exhale through the wearer's pharynx. The inner cannula includes a fourth port for orienting adjacent the first port, a fifth port for orienting adjacent the second port and a second passageway coupling the fourth port to the fifth port to permit the flow of gases from the fourth port to the fifth during inhalation by the wearer. A first valve controls flow through the third port. A second valve assumes a first orientation when the flow of respiration products through the third port is relatively less impeded and a second orientation permitting respiration products to flow from the second port to the first port and out the first port when flow through the third port is relatively more impeded.

### CROSS-REFERENCE TO RELATED APPLICATIONS

This is a related application to U.S. Ser. No. 11/318,649, filed Dec. 27, 2005 and published Jun. 28, 2007 as 2007/0144526. The disclosure of U.S. Ser. No. 11/318,649 is hereby incorporated herein by reference.

### FIELD OF THE INVENTION

This invention relates to improvements in tracheotomy tubes.

### BACKGROUND OF THE INVENTION

This invention is directed toward the problem of being unable to produce audible laryngeal voice, and thus, the inability to speak, that confronts individuals whose breathing is provided mechanically by a respirator which is connected to a cuffed tracheotomy tube inserted into the trachea of a wearer below the level of the vocal cords. The cuff on the tracheotomy tube is inflated, for example, with air, so that the cuff seals substantially fluid tight against the wall of the trachea. The purposes of the inflated cuff include: to protect against leakage of saliva and other secretions around the tracheotomy tube and into the lungs; and, to prevent the air being delivered under pressure from the respirator through the tracheotomy tube to the lungs and exhalation from the lungs from escaping around the tracheotomy tube and out through the mouth and nose of the wearer. In other words, the inflated cuff provides a closed mechanical respiratory system that completely bypasses the upper airway above the level of the tracheotomy tube, including the vocal cords. The side effects of this include the elimination of exhaled airflow upward through the vocal cords. Of course, this eliminates voice production by exhalation products from the lungs.

Currently, there are three available options for individuals being mechanically ventilated via a cuffed tracheotomy tube to produce audible voice and speech with their own vocal cords. The first of these options is described in O. Hessler, M. D., K. Rehder, M. D., and S. W. Karveth, M C, U.S.A., “Tracheostomy Cannula for Speaking During Artificial Respiration,” Anesthesiology, vol. 25, no. 5, pp. 719-721 (1964). There is no known commercially available device constructed as described in Hessler, et al.

The second option is a so-called “talking tracheotomy tube,” which is a conventional cuffed tracheotomy tube manufactured with an 8-10 French conduit extending along its length. The distal end of this conduit terminates above the level of the inflated cuff. The proximal end of this conduit is connected to a source of, for example, compressed air. Examples of such a device are manufactured by Sims Portex, Inc., and Bivona Surgical Inc. The wearer of such a device is able to stop and start the flow of compressed air to the distal end of this conduit, thereby enabling the stopping and starting of the flow of air upward through his or her vocal cords, enabling the wearer to produce speech. This speech airflow is completely independent of the respiratory airflow through the tracheotomy tube. Such talking tracheotomy tubes have been available for several years, but are not in widespread use, perhaps owing to numerous mechanical limitations.

The third option is systems of the types illustrated and described in U.S. Pat. No. 6,722,367 and U.S. Ser. No. 11/318,649, the disclosures of both of which are hereby incorporated herein by reference.

The following are also of interest: U.S. Pat. Nos. 3,688,774; 3,996,939; 4,211,234; 4,223,411; 4,280,492; 4,304,228; 4,449,523; 4,459,984; 4,573,460; 4,589,410; 4,596,248; 4,852,565; 5,056,515; 5,107,828; 5,217,008; 5,255,676; 5,297,546; 5,329,921; 5,339,808; 5,343,857; 5,349,950; 5,391,205; 5,392,775; 5,458,139; 5,497,768; 5,507,279; 5,515,844; 5,584,288; 5,599,333; RE35,595; 5,687,767; 5,688,256; 5,746,199; 5,771,888; 5,957,978; 6,053,167; 6,089,225; 6,102,038; 6,105,577; 6,135,111; 6,463,927; 6,814,007; foreign/international patent publications: DE 25 05 123; DE 37 20 482; DE 38 13 705; DE 195 13 831; WO 99/07428; WO 99/12599; WO 00/32262; other publications: Quick Reference Guide to Shiley's “Quality-Of Life” Line of Tracheostomy Products, 1991; Granuloma Associated with Fenestrated Tracheostomy Tubes, Padmanabhan Siddharth, MD, PhD, FACS and Lawrence Mazzarella, MD, FACS, Case Reports, vol. 150, August 1985, pp. 279-280; Technical Support Information Connections with the Passy-Muir Tracheostomy and Ventilator Speaking Valves, one sheet; Tracheostomy and Laryngectomy Tubes, pp. 568 and 572; Tracheostomy Tube Adult Home Care Guide, Shiley Tracheostomy Products, Mallinckrodt Medical pp. 1-40; D. Hessler, MD, K. Rehder, MD and S. W. Karveth, MD, “Tracheostomy Cannula for Speaking During Artificial Respiration”, Anesthesiology, vol. 25, No. 5, pp. 719-721 (1964). No representation is intended by this listing that a thorough search of all material prior art has been conducted, or that no better art than that listed is available. Nor should any such representation be inferred. The disclosures of all of the above are hereby incorporated herein by reference.

Unless he or she is wearing a device of the type illustrated and described in the above identified U.S. Pat. No. 6,722,367 or U.S. Ser. No. 11/318,649, a ventilator-dependent patient breathing through a cuffed tracheotomy tube is unable to produce audible voice with his or her vocal cords. This is so because without a device of the type illustrated and described in the above identified U.S. Pat. No. 6,722,367 or U.S. Ser. No. 11/318,649, the cuff of the tracheotomy tube he or she wears prevents exhalations from going around the lower end of the tube and upward through the vocal cords. This situation continues until the wearer's condition improves sufficiently that the cuff on the tracheotomy tube can be deflated so that exhaled air can pass around the tracheotomy tube and up through the wearer's vocal cords, mouth and nose, permitting audible vocal cord vibrations for speech.

The invention alleviates this situation. When coupled to a respirator with its cuff inflated, a valved, cuffed tracheotomy tube system according to the invention directs air on the inhalation cycle of the respirator to the lungs. Exhalations are directed by the valved, cuffed tracheotomy tube system according to the invention to the upper airway, permitting vocal cord vibration and audible laryngeal speech. The lungs of the wearer are protected against overinflation due to obstruction of the upper airway by a valve according to the invention.

### DISCLOSURE OF THE INVENTION

According to an aspect of the invention, an outer cannula has a first port for orienting outside the neck of a wearer, a second port for orienting within the trachea of the wearer, a first passageway coupling the first port to the second port to permit the flow of gases from the first port to the second during inhalation by the wearer and from the second port during exhalation by the wearer, and a third port between the first and second ports. An inner cannula is provided for insertion into the first passageway via the first port when the wearer desires to be able to exhale through the wearer's pharynx. The inner cannula includes a fourth port for orienting adjacent the first port, a fifth port for orienting adjacent the second port and a second passageway coupling the fourth port to the fifth port to permit the flow of gases from the fourth port to the fifth during inhalation by the wearer and from the fifth port during exhalation by the wearer. A first valve controls flow through the third port. The first valve assumes a first orientation to permit flow from the first port to the second port when the first port is at a higher pressure than the second port and a second orientation to permit flow from the second port when the second port is at a higher pressure than the first port. A second valve assumes a first orientation when the flow of respiration products through the third port is relatively less impeded and a second orientation permitting respiration products to flow from the second port to the first port and out the first port when flow through the third port is relatively more impeded.

According to another aspect of the invention, an outer cannula has a first port for orienting outside the neck of a wearer, a second port for orienting within the trachea of the wearer, a first passageway coupling the first port to the second port to permit the flow of gases from the first port to the second during inhalation by the wearer and from the second port during exhalation by the wearer, and a third port between the first and second ports. An inner cannula is provided for insertion into the first passageway via the first port when the wearer desires to be able to exhale through the wearer's pharynx. The inner cannula includes a fourth port for orienting adjacent the first port, a fifth port for orienting adjacent the second port and a second passageway coupling the fourth port to the fifth port to permit the flow of gases from the fourth port through the fifth during inhalation by the wearer and preventing the flow of gases from the fourth port during exhalation by the wearer. A first valve controls flow through the third port. The first valve assumes a first orientation to permit flow from the fourth port to the fifth port when the fourth port is at a higher pressure than the fifth port and a second orientation to prevent flow from the fourth port when the fifth port is at a higher pressure than the fourth port. A second valve assumes a first orientation when the flow of respiration products through the third port is relatively less impeded and a second orientation permitting respiration products to flow from the second port to the first port and out the first port when flow through the third port is relatively more impeded.

Illustratively, the first valve includes a resilient region which lies adjacent the third port when the inner cannula is properly oriented within the outer cannula. The inner cannula further includes a third valve operatively associated with the inner cannula and a region between the resilient region and the third valve which provides a passageway between the inner cannula and the outer cannula when the inner cannula is properly oriented within the outer cannula.

Further illustratively, an inflatable cuff is formed on the outer cannula between the second port and the third port. A first conduit extends from adjacent the first port to the cuff for introducing an inflating fluid into the cuff when it is desired to inflate the cuff and removing inflating fluid from the cuff when it is desired to deflate the cuff. The inner cannula includes a second conduit to evacuate a region of a trachea of a wearer adjacent the cuff. The second conduit includes an opening which lies adjacent the closest point in the third port to the cuff when the inner cannula is in a use orientation in the outer cannula.

Further illustratively, an inflatable cuff is formed on the outer cannula between the second port and the third port. The inflatable cuff is formed by a sleeve including a first end, a second end and a third region between the first and second ends. The sleeve is located around the outer cannula with at least the first end of the sleeve between the outer cannula and the third region of the sleeve. A conduit extends from a first end of the outer cannula to the cuff for introducing an inflating fluid into the cuff when it is desired to inflate the cuff and removing inflating fluid from the cuff when it is desired to deflate the cuff.

Further illustratively, an inflatable cuff is formed on the outer cannula between the second port and the third port. A conduit extends from adjacent the first port to the cuff for introducing an inflating fluid into the cuff when it is desired to inflate the cuff and removing inflating fluid from the cuff when it is desired to deflate the cuff. The third port is oriented immediately adjacent the cuff to permit the flow of gas from inside the outer cannula through the third port and out of the tracheotomy tube.

### BRIEF DESCRIPTIONS OF THE DRAWINGS

The invention may best be understood by referring to the following detailed description and accompanying drawings. In the drawings:

FIG. 1 illustrates a fragmentary sectional side elevational view of the upper trachea, lower pharynx and front of the neck of a wearer of a device according to the invention during inspiration of air into the lungs of the wearer under the control of a ventilator;

FIG. 1a illustrates a perspective view of a detail of the device illustrated in FIG. 1 in the orientation illustrated in FIG. 1;

FIG. 2 illustrates a fragmentary sectional side elevational view of the upper trachea, lower pharynx and front of the neck of a wearer of the device illustrated in FIG. 1 during normal expiration from the lungs of the wearer upward through the pharynx of the wearer and out the nose and/or mouth of the wearer;

FIG. 3 illustrates a fragmentary sectional side elevational view of the upper trachea, lower pharynx and front of the neck of a wearer of the device illustrated in FIGS. 1-2 during expiration from the lungs of the wearer back through the device illustrated in FIGS. 1-2 and the ventilator, owing to an obstruction (not shown) of the upper airway of the wearer leading to the nose and mouth; and,

FIG. 3a illustrates a perspective view of a detail of the device illustrated in FIGS. 1-3 in the orientation illustrated in FIG. 3.

### DETAILED DESCRIPTIONS OF ILLUSTRATIVE EMBODIMENTS

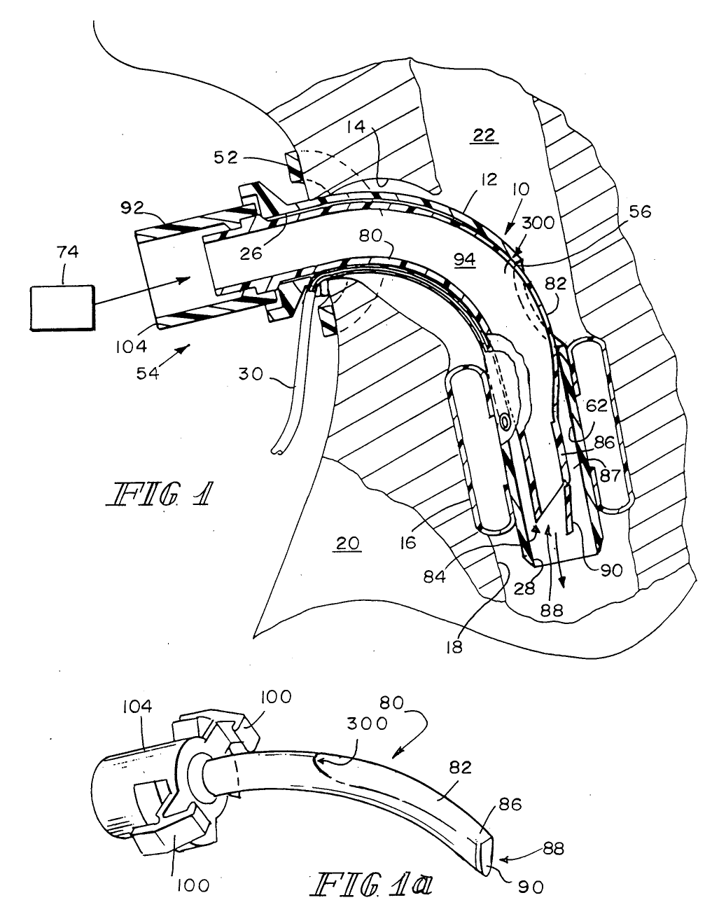

Referring now particularly to FIG. 1, a speaking tracheotomy tube system 10 includes an outer cannula 12 for insertion into a tracheostoma 14. Outer cannula 12 includes an inflatable cuff 16. Cuff 16 lies in the trachea 18 of a wearer 20 below the passageway 22 upward into the pharynx 24 of the wearer 20. Outer cannula 12 also includes a first port 26 which resides outside the neck of the wearer 20 during use and a second port 28 which resides inside the neck of the wearer 20 below cuff 16 during use. The cuff 16 is inflatable through a line 30 once the outer cannula 12 is in place in the trachea 18 to minimize the passage of secretions from the upper respiratory tract, including pharynx 24, downward into the lungs of the wearer 20. Such secretions pool above the cuff 16 when the cuff 16 is inflated in place, and may be evacuated as illustrated and described in U.S. Ser. No. 11/318,649.

The outer cannula 12 includes a pivotally mounted attachment plate 52 adjacent its proximal end 54 to facilitate attachment, for example, by a strap or belt around the neck of the wearer 20. The outer cannula 12 also includes a fenestration 56 which permits the wearer 20 to speak by providing a flow of exhaled respiratory gases upward through the fenestration 56 and into the pharynx 24. Speech may then be articulated in accordance with known principles. Although only one such fenestration 56 is illustrated, it should be understood that any number of fenestrations 56 may be provided in the outer cannula 12 for this purpose.

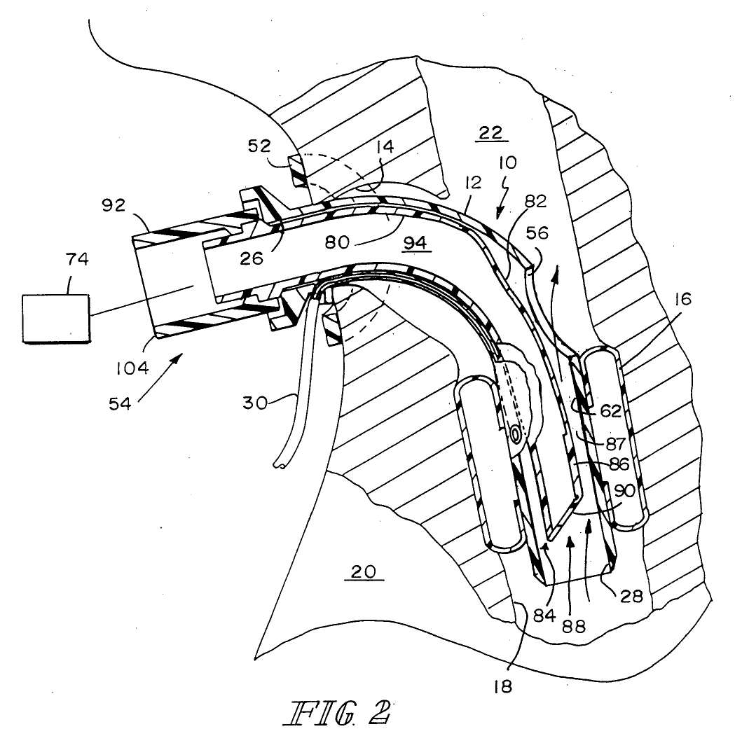

During times when the wearer desires to speak, a speaking inner cannula 80 is inserted into the outer cannula 12. Speaking inner cannula 80 includes a flexible, balloon-like region 82 adjacent fenestration 56, a region 86 between region 82 and lower end 84 which provides a passageway 87 between region 86 and the inner sidewall of lumen 62 of outer cannula 12, 12′, and a valve 88 including a resilient flap 90 at its lower end 84. Speaking inner cannula 80 functions in the following manner when it is inserted into outer cannula 12, locked in place and its outer end 92 attached to a ventilator 74. Referring first to FIG. 1, during pressurization by the ventilator 74, balloon-like region 82 inflates, sealing against fenestration 56 and preventing the escape of ventilator-provided air upward through fenestration 56 and the wearer 20's pharynx 24. Flap 90 of valve 88 opens, permitting air to flow into the lungs of the wearer 20. Referring now to FIG. 2, during exhalation, the ventilator 74 removes pressure at the outer end 92 of speaking inner cannula 80. The flap 90 of valve 88 closes, closing the lumen 94 of speaking inner cannula 80 against the passage upward of respiratory products through speaking inner cannula 80, permitting the balloon-like region 82 to deflate somewhat and opening passageway 87 upward from the lungs of the wearer 20 through fenestration 56. Respiratory products in the wearer 20's lungs escape upward through passageway 87, through fenestration 56, and are released into the wearer's pharynx 24, providing sufficient flow to permit the wearer 20 to speak.

Speaking inner cannula 80 is releasably fixed to the outer cannula 12 by locking tabs 100 formed with the coupler 104 of cannula 80 by which cannula 80 is coupled to ventilator 74. The tabs 100 are flexibly formed to effect the appropriate orientation of the balloon-like region 82 of speaking inner cannula 80 with respect to fenestration(s) 56 when speaking inner cannula 80 is inserted into outer cannula 56.

Speaking inner cannula 80 is provided with an additional valve 300 formed by a somewhat C-shaped slit at the similarly somewhat C-shaped transition region between the proximal end of thinner walled, more flexible balloon-like region 82 and the somewhat thicker walled, less flexible upper region of the inner cannula 80. When speaking inner cannula 80 is in place in outer cannula 12, valve 300 lies adjacent but proximally, that is toward ventilator 74, of fenestration 56.

As noted above, and with reference again to FIG. 1, in use, during the inhalation portion of the ventilator 74 cycle, air is pumped by the ventilator 74 through inner cannula 80 into the lungs of the wearer 20. Pressurization of inner cannula 80 inflates balloon-like region 82 against the inner sidewall of outer cannula 12, closing fenestration 56, thus preventing the escape of air upward through the pharynx 24 of the wearer 20. Additionally, valve 300 is sealed against the inner sidewall of outer cannula 12 by this same inflation pressure. The flap 90 of valve 88 opens, and the air flows into the lungs of the wearer 20. With reference again to FIG. 2, then during the exhalation portion of the ventilator 74 cycle, pressure at the outer end 92 of outer cannula 12 is removed. Flap 90 of valve 88 closes, balloon-like region 82 deflates away from fenestration 56, opening fenestration 56. Respiratory products flow upward past balloon-like region 82, through fenestration 56, and upward through the pharynx 24 of the wearer 20 permitting speech.

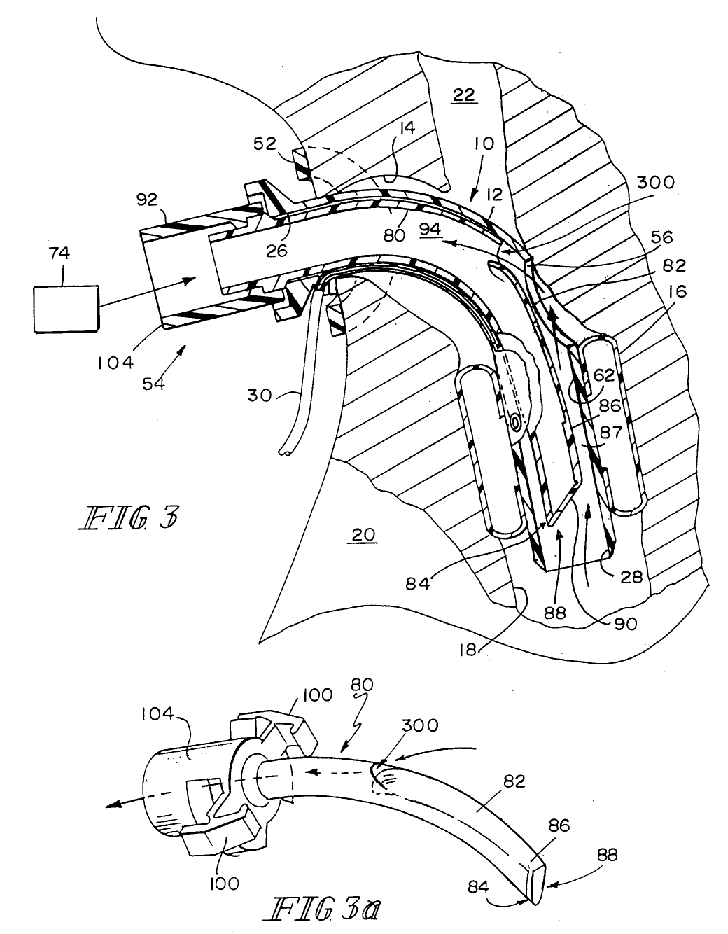

With reference now to FIG. 3, there is a remote possibility that the upper airway through the pharynx 24 of the wearer 20 can become obstructed, for example, by secretions or the like which the wearer 20 is unable to develop sufficient coughing pressure to expel, by mispositioning or misfitting of outer cannula 12, or other causes. Thus, there is a remote possibility that the next inhalation cycle of the ventilator 74 will begin without exhalation products from the previous exhalation cycle having been expelled. In this event, a second inhalation cycle of the ventilator 74 will pump the next volume of air through inner cannula 80 and into the lungs of the wearer 24 as described above. After this second inhalation cycle, the lungs of the wearer 20 have become somewhat overinflated. The lungs of the wearer 20 and the wearer 20's upper trachea will have become sufficiently pressurized that valve 300 will open by deflection of valve 300 flap inward toward the center of inner cannula 80 and away from the inner wall of outer cannula 12 to permit respiration products to return back through the proximal end 92 of cannula 12 and the ventilator 74 to relieve the excess pressure on the lungs of the wearer 20 and prevent overinflation damage to the lungs of the wearer 20.

What is claimed is:

1. In combination, an outer cannula having a first port for orienting outside the neck of a wearer, a second port for orienting within the trachea of the wearer and a first passageway coupling the first port to the second port to permit the flow of gases from the first port to the second during inhalation by the wearer and from the second port during exhalation by the wearer, a third port between the first and second ports, and an inner cannula for insertion into the first passageway via the first port when the wearer desires to be able to exhale through the wearer's pharynx, the inner cannula including a fourth port for orienting adjacent the first port, a fifth port for orienting adjacent the second port and a second passageway coupling the fourth port to the fifth port to permit the flow of gases from the fourth port to the fifth during inhalation by the wearer and from the fifth port during exhalation by the wearer, a first valve controlling flow through the third port, the first valve assuming a first orientation to permit flow from the first port to the second port when the first port is at a higher pressure than the second port, and a second orientation to permit flow from the second port when the second port is at a higher pressure than the first port, and a second valve assuming a first orientation when the flow of respiration products through the third port is relatively less impeded and a second orientation permitting respiration products to flow from the second port to the first port and out the first port when flow through the third port is relatively more impeded.

2. The apparatus of claim 1 wherein the first valve includes a resilient region which lies adjacent the third port when the inner cannula is properly oriented within the outer cannula, the inner cannula further including a third valve operatively associated with the inner cannula and a region between the resilient region and the third valve which provides a passageway between the inner cannula and the outer cannula when the inner cannula is properly oriented within the outer cannula.

3. The apparatus of claim 1 further including an inflatable cuff formed on the outer cannula between the second port and the third port, a first conduit extending from adjacent the first port to the cuff for introducing an inflating fluid into the cuff when it is desired to inflate the cuff and removing inflating fluid from the cuff when it is desired to deflate the cuff, the inner cannula including a second conduit to evacuate a region of a trachea of a wearer adjacent the cuff, the second conduit including an opening which lies adjacent the closest point in the third port to the cuff when the inner cannula is in a use orientation in the outer cannula.

4. The apparatus of claim 1 further including an inflatable cuff formed on the outer cannula between the second port and the third port, the inflatable cuff formed by a sleeve including a first end, a second end, and a third region between the first and second ends, the sleeve located around the outer cannula with at least the first end of the sleeve between the outer cannula and the third region of the sleeve, and a conduit extending from a first end of the outer cannula to the cuff for introducing an inflating fluid into the cuff when it is desired to inflate the cuff and removing inflating fluid from the cuff when it is desired to deflate the cuff.

5. The apparatus of claim 1 further including an inflatable cuff formed on the outer cannula between the second port and the third port, a conduit extending from adjacent the first port to the cuff for introducing an inflating fluid into the cuff when it is desired to inflate the cuff and removing inflating fluid from the cuff when it is desired to deflate the cuff, the third port oriented immediately adjacent the cuff to permit the flow of gas from inside the outer cannula through the third port and out of the tracheotomy tube.

6. In combination, an outer cannula having a first port for orienting outside the neck of a wearer, a second port for orienting within the trachea of the wearer and a first passageway coupling the first port to the second port to permit the flow of gases from the first port to the second during inhalation by the wearer and from the second port during exhalation by the wearer, a third port between the first and second ports, and an inner cannula for insertion into the first passageway via the first port when the wearer desires to be able to exhale through the wearer's pharynx, the inner cannula including a fourth port for orienting adjacent the first port, a fifth port for orienting adjacent the second port and a second passageway coupling the fourth port to the fifth port to permit the flow of gases from the fourth port through the fifth during inhalation by the wearer and preventing the flow of gases from the fourth port during exhalation by the wearer, a first valve controlling flow through the third port, the first valve assuming a first orientation to permit flow from the fourth port to the fifth port when the fourth port is at a higher pressure than the fifth port, and a second orientation to prevent flow from the fourth port when the fifth port is at a higher pressure than the fourth port, and a second valve assuming a first orientation when the flow of respiration products through the third port is relatively less impeded and a second orientation permitting respiration products to flow from the second port to the first port and out the first port when flow through the third port is relatively more impeded.

7. The apparatus of claim 6 wherein the first valve includes a resilient region which lies adjacent the third port when the inner cannula is properly oriented within the outer cannula, the inner cannula further including a third valve operatively associated with the inner cannula and a region between the resilient region and the third valve which provides a passageway between the inner cannula and the outer cannula when the inner cannula is properly oriented within the outer cannula.

8. The apparatus of claim 6 further including an inflatable cuff formed on the outer cannula between the second port and the third port, a first conduit extending from adjacent the first port to the cuff for introducing an inflating fluid into the cuff when it is desired to inflate the cuff and removing inflating fluid from the cuff when it is desired to deflate the cuff, the inner cannula including a second conduit to evacuate a region of a trachea of a wearer adjacent the cuff, the second conduit including an opening which lies adjacent the closest point in the third port to the cuff when the inner cannula is in a use orientation in the outer cannula.

9. The apparatus of claim 6 further including an inflatable cuff formed on the outer cannula between the second port and the third port, the inflatable cuff formed by a sleeve including a first end, a second end, and a third region between the first and second ends, the sleeve located around the outer cannula with at least the first end of the sleeve between the outer cannula and the third region of the sleeve, and a conduit extending from a first end of the outer cannula to the cuff for introducing an inflating fluid into the cuff when it is desired to inflate the cuff and removing inflating fluid from the cuff when it is desired to deflate the cuff.

10. The apparatus of claim 6 further including an inflatable cuff formed on the outer cannula between the second port and the third port, a conduit extending from adjacent the first port to the cuff for introducing an inflating fluid into the cuff when it is desired to inflate the cuff and removing inflating fluid from the cuff when it is desired to deflate the cuff, the third port oriented immediately adjacent the cuff to permit the flow of gas from inside the outer cannula through the third port and out of the tracheotomy tube.
